(19) United States
(12) Patent Application Publication
(10) Pub. No.: US 2017/0354756 A1
(43) Pub. Date: Dec. 14, 2017

(54) POLYPHOSPHATE HYDROGELS AND METHODS OF MAKING AND USING THEREOF

(71) Applicants: Russell J. STEWART, Salt Lake City, UT (US); Dwight LANE, Salt Lake City, UT (US); UNIVERSITY OF UTAH RESEARCH FOUNDATION, Salt Lake City, UT (US)

(72) Inventors: Russell J. STEWART, Salt Lake City, UT (US); Dwight LANE, Salt Lake City, UT (US)

(73) Assignee: UNIVERSITY OF UTAH RESEARCH FOUNDATION, Salt Lake City, UT (US)

(21) Appl. No.: 15/527,431

(22) PCT Filed: Nov. 18, 2015
(86) PCT No.: PCT/US15/61226

§ 371 Date: May 17, 2017

Related U.S. Application Data
(60) Provisional application No. 62/081,473, filed on Nov. 18, 2014.
(60) Provisional application No. 62/081,051, filed on Nov. 18, 2014.

(51) Int. Cl. A61L 27/16; A61K 31/7036; A61L 27/54; A61L 27/52; C08J 3/075; A61F 2/02

(52) U.S. Cl. CPC A61L 27/16; A61L 27/52; C08J 3/075; A61K 31/7036; A61L 27/54; A61F 2/02; C08J 2333/14; A61L 2300/406

(57) ABSTRACT

Described herein are hydrogels with improved mechanical properties. The hydrogels are composed of two polymer networks covalently crosslinked with one another. The addition of a multivalent cation and/or polycation to the hydrogels further crosslinks the polyphosphate network and can modulate the mechanical properties of the hydrogels as needed. Methods for making and using the hydrogels described herein are presented below.

### CROSS REFERENCE TO RELATED APPLICATIONS

This application claims priority upon U.S. provisional application Serial Nos. 62/081,051 and 62/081,473, both filed Nov. 18, 2014. These applications are hereby incorporated by reference in their entirety.

### ACKNOWLEDGEMENTS

This invention was made with government support under W911NF-13-1-0319 awarded by Army Research Office. The government has certain rights in the invention.

### BACKGROUND

Despite considerable progress in tissue engineering approaches to regenerate damaged or worn-out soft structural tissues, there likely will always be a need for inert, biocompatible, synthetic replacement materials. Progress has been limited, though, because the structure and mechanical properties of conventional hydrogels have little resemblance to the exquisite hierarchical organization, strength, toughness, and graded mechanics of natural tissues. One drawback with current hydrogels is that they are brittle and fracture at low strains. The usefulness of traditional synthetic hydrogels is also limited by their propensity to swell in watery environments, which further degrades their mechanical attributes. Thus, there is a need for new hydrogels with improved mechanical properties.

### SUMMARY

Described herein are hydrogels with improved mechanical properties. The hydrogels are composed of two polymer networks covalently crosslinked with one another. The addition of a multivalent cation and/or polycation to the hydrogels further crosslinks the polyphosphate network and can modulate the mechanical properties of the hydrogels as needed. Methods for making and using the hydrogels described herein are presented below.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows an exemplary synthesis of producing the hydrogels described herein.

FIG. 2 shows hydrogel volume change during metal ion exchange. The hydrogels contained 6.5 wt/vol % pMOEP and 1.0 wt/vol % pAAm for a total polymer concentration of 7.5 wt/vol % before deswelling by the addition of divalent metal ions. For each data point n=3 and error bars=±1 s.d.

FIGS. 3A and 3B show the critical pMOEP concentration dependence of Ca²⁺-hydrogel toughening. (A) Representative stress strain curves for hydrogels prepared with increasing pMOEP and decreasing pAAm concentrations. The total polymer concentration was fixed at 7.5 wt/vol %. Ovals represent the area enclosed by ±1 s.d. of the mean stress and elongation for each hydrogel formulation (n≧3). (B) The equilibrium volume of the hydrogels declined with increasing pMOEP/pAAm wt % ratio. The initial modulus and yield stress had a non-linear dependence on pMOEP/pAAm wt % ratio. Error bars represent ±1 s.d., n≧3.

FIG. 4 shows the spontaneous recovery of initial length of a Ca^{2±}-hydrogel strained to 90% underwater. Scale bar=6 mm.

FIGS. 5A-5C show the recovery kinetics of divalent metal ion-equilibrated hydrogels. (A) Representative stress strain profiles with increasing recovery periods between cycles. Grey curves: Ca²⁺. Green curves: Mg²⁺. (B) Time course of initial modulus and yield stress recovery. (C) Time course of hysteresis and initial length recovery. Error bars=±1 s.d., n≧3.

FIGS. 6A and 6B show strain rate dependence of Ca²-hydrogel stress response. (A) Representative stress strain curves of cyclically loaded hydrogels. (B) Semi-log plot of yield stress as a function of strain rate. Dashed line is best linear fit. Error bars=±1 s.d., n≧3.

FIG. 7 shows the stress response during strain to fracture for hydrogels equilibrated with Na⁺, Mg²⁺, Ca²⁺, and Zn²⁺. Ellipses represent the mean±1 s.d. Inset: Expanded scale to accent Mg²⁺ and Na⁺ hydrogel stress response.

FIGS. 8A-8D show normalized ATR-FTIR spectra in the region corresponding to P—O⁻ vibrational modes of metal ion equilibrated hydrogels at pH 8.0 (blue shaded peaks). (A) Na⁺-equilibrated hydrogels. (B) Ca⁺-equilibrated hydrogels. (C) Mgt equilibrated hydrogels. (D) Zn⁺-equilibrated hydrogels. The vertical numbers are the area of the fit peak (dotted spectra) in normalized absorption units.

FIG. 9 shows an exemplary adhesive hydrogel described herein.

FIG. 10 shows an example of an adhesive hydrogel described herein applied to a substrate.

FIG. 11 shows the percent volume decrease in the hydrogel after the addition of 5 mM Tobramycin. The pH is 12 for the first two hours and then 7.5 for all subsequent time points (n=3).

FIG. 12 shows the cumulative release of tobramycin from 0.02 ml hydrogels (n=3).

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a monomer” includes mixtures of two or more such monomers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight/volume percent of the hydrogel or a component used to produce the hydrogel, unless specifically stated to the contrary, is the amount of polymer or component in grams per 100 mL. For example, a hydrogel that is 7.5 wt/vol % is 7.5 g of polymer in 100 mL of hydrogel before the addition of multivalent metal ions or polycations.

“Subject” refers to mammals including, but not limited to, humans, non-human primates, sheep, dogs, rodents (e.g., mouse, rat, etc.), guinea pigs, cats, rabbits, cows, and non-mammals including chickens, amphibians, and reptiles.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “phenolic group” as used herein is any carbon-based aromatic group including, but not limited to, an aryl group possessing one or more hydroxyl groups covalently bonded to the aryl group

The term “aryl group” as used herein is any carbon-based aromatic group possessing at least one benzene ring. The aryl group can possess a single benzene ring or two or more benzene rings either fused (e.g., naphthalene) or covalently bonded together by a single bond. The term “aryl group” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “lower alkyl” as used herein is an alkyl group having 1 to 5 carbon atoms. The alkyl group can be branched or straight chain.

The term “hydroxyalkyl” as used herein is an alkyl groups having one or more hydroxyl groups covalently bonded to it. The alkyl group can be branched or straight chain having from 1 to 10 carbon atoms. For example, with 2-hydroxyethyl methacrylate (HEMA), the —CH₂CH₂OH group is the hydroxyalkyl group. A “hydroxyl-substituted (lower alkyl)” has one or more hydroxyl groups covalently bonded to an alkyl group having one to five carbon atoms. HEMA is an example of a hydroxyl-substituted (lower alkyl)methacrylate.

A hydroxylalkyl acrylamide and a hydroxylalkyl methacrylamide is acrylamide and methacrylamide, respectively, where one of the —NH₂ protons is substituted with a hydroxyalkyl group.

A (lower alkyl)acrylamide and a (lower alkyl)methacrylamide is acrylamide and methacrylamide, respectively, where one of the —NH₂ protons is substituted with a lower alkyl group.

A (meth)acrylic monomer used to produce the first polymeric network is any compound that includes an acryloyl group or a methacryloyl group as depicted in the formula below

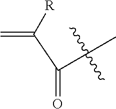

where when R is hydrogen, it is an acryloyl group and when R is methyl it is a methacryloyl group.

Described herein are hydrogels with improved mechanical properties. The hydrogels are composed of two polymer networks covalently crosslinked with one another.

In one aspect, the hydrogel comprises (a) a first polymeric network comprising a polymer derived from a (meth)acrylic monomer; (b) a second polymeric network comprising a polyanion, wherein the first polymeric network and second polymeric network are covalently crosslinked with each other, and (c) a plurality of multivalent cations, a polycation, or a combination thereof that non-covalently crosslinks the second polymeric network. The components used to produce the hydrogels described herein as well as their applications thereof are provided below.

In one aspect, the hydrogels are produced by
a. Polymerizing a (meth)acrylic monomer to produce a first polymeric network in the presence of (1) a second polymeric network comprising a polyphosphate prepolymer comprising a plurality of phosphate groups and a plurality pendant acryloyl groups, pendant methacryloyl groups, or a combination thereof, and (2) a free radical initiator, wherein the first network and second network are covalently crosslinked with each other to produce a first hydrogel; and
b. contacting the first hydrogel with a multivalent cation, a polycation, or a combination thereof to further non-covalently crosslink the second polymeric network.

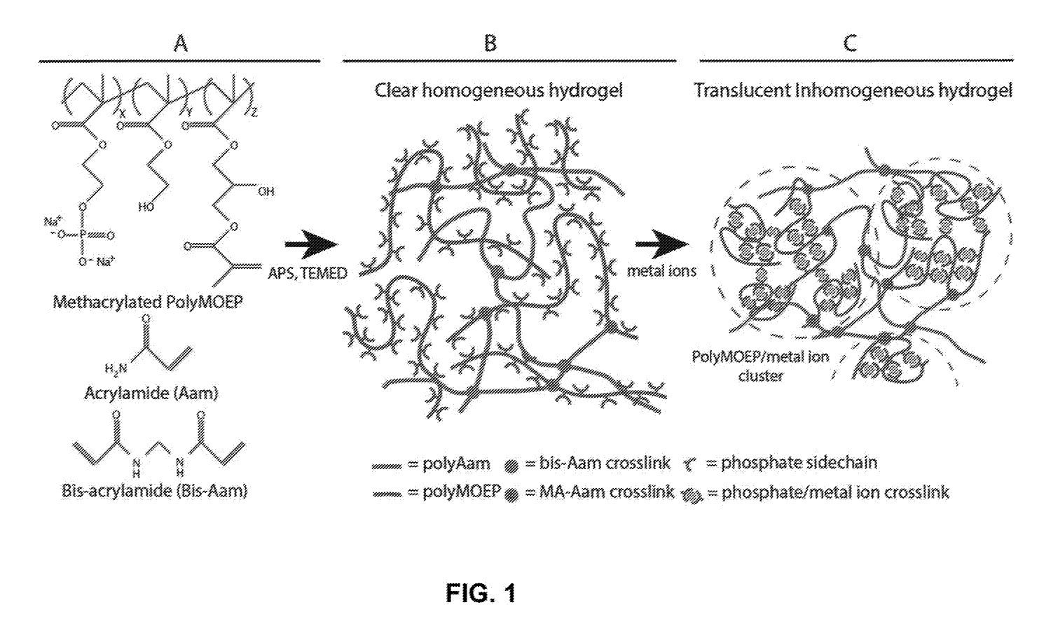

There are two types of crosslinks in the hydrogels. First, covalent crosslinking occurs between the two polymer networks present in the hydrogel. The second type of crosslinking involves the interaction (i.e., non-covalent crosslinking) between the phosphate groups in the polyphosphate network and the multivalent cations or polycation. The interaction between the phosphate groups and the multivalent cations or polycation can involve electrostatic bonding, ionic bonding, or coordination bonding. A non-limiting example of the two types of crosslinking is depicted in FIG. 1.

Step (a) for producing the hydrogels generally involves admixing one or more (meth)acrylic monomers, the polyphosphate prepolymer, and the initiator in a solvent. In one aspect, the solvent is water or a buffered water solution typically used in biological applications (e.g., TRIS, TAPS, TAPSO, HEPES, TES, MOPS). The pH of the solution composed of the one or more (meth)acrylic monomers, the polyphosphate prepolymer, and the initiator can also vary. In general, the pH is high enough to ionize the phosphate groups present in the polyphosphate prepolymer. Upon polymerization of the (meth)acrylic monomer, a first polymeric network is produced. During the polymerization, the first polymeric network can covalently crosslink with the polyphosphate prepolymer (i.e., the second polymeric network), as the polyphosphate prepolymer has a plurality pendant acryloyl groups, pendant methacryloyl groups, or a combination thereof that can covalently crosslink with the acryloyl groups and/or methacryloyl groups present on the first polymeric network. As will be discussed below, an optional crosslinker can be added during step (a) in order to further covalently crosslink the first and second polymeric networks.

In other aspect, the hydrogels can be molded into any desired shape and size as needed. For example, the components in step (a) can be poured into a mold, the components subsequently polymerized to produce the hydrogel having a specific size and dimensions. The molded article can then be subsequently contacted with the multivalent cations and/or polycation. Exemplary procedures for producing molded article composed of hydrogels are provided in the Examples.

The total amount of the polymer in the hydrogel produced in step (a) (i.e., the sum of the first and second polymeric network) can vary from 1 to 20 wt/vol %. The weight ratio of the first to the second polymeric networks present in the hydrogel produced in step (a) can vary from 1 to 99%.

Examples of (meth)acrylic monomers useful herein include, but are not limited to, acrylic acid, methacrylic acid, hydroxyalkyl methacrylate, a hydroxyalkyl acrylate, acrylamide, methacrylamide, a (lower alkyl)acrylamide, a (lower alkyl)methacrylamide, hydroxyl-substituted (lower alkyl)acrylate, a hydroxyl-substituted (lower alkyl)methacrylate, a hydroxylalkyl acrylamide, a hydroxylalkyl methacrylamide, a hydroxyl-substituted (lower alkyl)acrylamide, a hydroxyl-substituted (lower alkyl)methacrylamide, or any combination thereof. In one aspect, the (meth)acrylic monomer is acrylamide or methacrylamide.

The second network includes a polyphosphate prepolymer having a plurality of phosphate groups and a plurality of pendant acryloyl groups, pendant methacryloyl groups, or a combination thereof.

In one aspect, the polyphosphate prepolymer is a polyacrylate having a plurality of pendant phosphate groups. For example, the polyphosphate prepolymer can be derived from the polymerization of (meth)acrylic monomers including, but not limited to, acrylates, methacrylates, and the like. In other aspects, the polyphosphate prepolymer is a random co-polymer, where segments or portions of the co-polymer possess phosphate groups and neutral groups depending upon the selection of the monomers used to produce the co-polymer. The polyphosphate prepolymer useful herein can be the free acid, a salt thereof, or a combination thereof depending upon reaction conditions (e.g., pH) used to produce the hydrogel.

In one aspect, the polyphosphate prepolymer is produced by (1) polymerizing a a phosphate (meth)acrylic monomer to produce a first polymer, and (2) grafting acryloyl groups, methacryloyl groups, or a combination thereof to the first polymer. A phosphate (meth)acrylic monomer is any acrylic monomer as defined herein having at least one phosphate group covalently bonded to the monomer.

In this aspect, any of the (meth)acrylic monomers discussed above can be copolymerized with a phosphate acrylic monomer to produce the polyphosphate prepolymer that can subsequently be modified with an acryloyl or methacryloyl group can be used in this embodiment. In one aspect, the phosphate (meth)acrylic monomer has the formula I

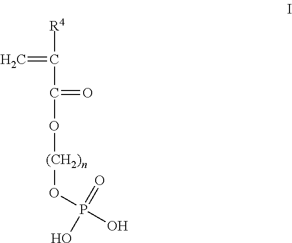

wherein R⁴ is hydrogen or an alkyl group, and n is from 1 to 10. In one aspect, R⁴ is methyl and n is 2 in formula I.

In another aspect, phosphate (meth)acrylic monomer of formula I is polymerized with acrylic acid, methacrylic acid, hydroxyalkyl methacrylate, hydroxyalkyl acrylate, acrylamide, methacrylamide, a (lower alkyl)acrylamide, a (lower alkyl)methacrylamide, a hydroxyl-substituted (lower alkyl)acrylamide, a hydroxyl-substituted (lower alkyl)methacrylamide, or any combination thereof.

In another aspect, the phosphate (meth)acrylic monomer of formula I is copolymerized with acrylic acid or methacrylic acid alone or in combination with one or more additional monomers such as a hydroxyalkyl methacrylate or hydroxyalkyl acrylate. After copolymerization, acryloyl groups and/or methacryloyl groups are grafted to the phosphate copolymer. In one aspect, when the phosphate copolymer possesses groups such as hydroxyl, carboxyl, or amino groups the can react with compounds that possess an acryloyl group or methacryloyl group. For example, glycidyl methacrylate can be used to graft methacryloyl groups on the phosphate copolymer. Using this approach acryloyl groups and/or methacryloyl groups are pendant to the polyphosphate copolymer backbone. This is depicted in FIG. 1, where for the polyphosphate prepolymer (methacrylated polyMOEP) both phosphate groups and methacrylate groups are pendant to the copolymer backbone. The Examples provide non-limiting procedures for making the phosphate copolymer as well as grafting acryloyl or methacryloyl groups on the first polymer to produce the polyphosphate prepolymer.

When the phosphate (meth)acrylic monomer of formula I polymerized with one or more (meth)acrylic monomers described above is used to produce the polyphosphate prepolymer, the resulting polyphosphate prepolymer will have a plurality of units of the formula II

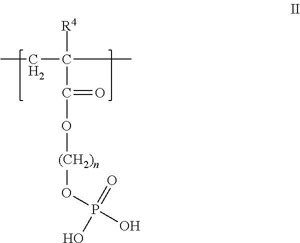

where R⁴ and n are defined above.

In one aspect, the polyphosphate prepolymer has from 20 mol % to 90 mol % of the units of formula II relative to the other monomers used to produce the polyphosphate prepolymer. For example, when the polyphosphate prepolymer is the polymerization product of a phosphate (meth)acrylic monomer of formula I, methacrylic acid, and 2-hydroxyethyl methacrylate, the amount of phosphate (meth)acrylic monomer of formula I can be from 20 mol % to 90 mol %, the amount of methacrylic acid can be from 1 mol % to 30 mol %, and the amount of 2-hydroxyethyl methacrylate is from 1 mol % to 30 mol %. In one aspect, the polyphosphate prepolymer has from 30 mol % to 90 mol %, 40 mol % to 90 mol %, or 50 mol % to 70 mol % of the units of formula II. In another aspect, the polyphosphate prepolymer has 20 mol %, 25 mol %, 30 mol %, 35 mol %, 40 mol %, 45 mol %, 50 mol %, 55 mol %, 60 mol %, 65 mol %, 70 mol %, 75 mol %, 80 mol %, 85 mol % or 90 mol % of the units of formula II, where any value can form a lower and upper end-point of a range.

In one aspect, the polyphosphate prepolymer is a random copolymer having the units depicted in formula III

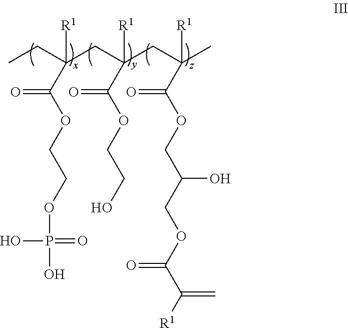

wherein x is from 40 to 90 mol %, y is from 1 to 30 mol %; and z is from 1 to 30 mol % of the polyphosphate prepolymer; and
each R¹ is independently hydrogen or methyl.

In one aspect, x (mol %) is 20, 25, 30, 35, 40, 45, 50, 55, 60, 65, 70, 75, 80, 85 or 90; y (mol %) is 1, 5, 10, 15, 20, 25, or 30; and z (mol %) is 1, 5, 10, 15, 20, 25, or 30, where any value can form a lower and upper end-point of a range for x, y, and z. In another aspect, x is from 50 to 70 mol %; y is from 5 to 20 mol %; and z is from 20 to 30 mol % of the polyphosphate prepolymer, and each R¹ is methyl. In another aspect, the polyphosphate prepolymer has a molecular weight of 1,000 Da to 200,000 Da, 25,000 Da to 100,000 Da, or 50,000 Da to 100,000 Da.

In other aspects, other polymers besides polyphosphate prepolymers can be used to produce the hydrogels. For example, the monomer having the formula I above can be substituted with the monomer of formula IV

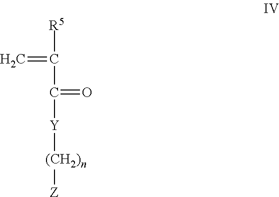

wherein R⁵ is hydrogen or an alkyl group;
n is from 1 to 10;
Y is oxygen, sulfur, or NR⁶, wherein R⁶ is hydrogen, an alkyl group, or an aryl group;
Z is sulfate, sulfonate, carboxylate, borate, boronate, or a phosphonate.

Free radical initiators typically used in the art for free radical polymerization can be used in step (a) above. In one aspect, the initiator includes organic peroxides or azo compounds. Examples of organic peroxides include ketone peroxides, peroxyketals, hydroperoxides, dialkyl peroxides, diacyl peroxides, peroxydicarbonates, peroxyesters, and the like.

Some specific non-limiting examples of azo compounds that can be used as the oil soluble initiator include: 2,2′-azobis-isobutyronitrile, 2,2′-azobis-2,4-dimethylvaleronitrile, 1,1′-azobis-1-cyclohexane-carbonitrile, dimethyl-2,2′-azobisisobutyrate, 1,1′-azobis-(1-acetoxy-1-phenylethane), 4,4′-azobis(4-cyanopentanoic acid) and its soluble salts (e.g., sodium, potassium), and the like.

In another aspect, the free radical initiator is a water-soluble initiator including, but not limited to, potassium persulfate, ammonium persulfate, sodium persulfate, and mixtures thereof. In another aspect, the initiator is an oxidation-reduction initiator such as the reaction product of the above-mentioned persulfates and reducing agents such as sodium metabisulfite and sodium bisulfite; and 4,4′-azobis(4-cyanopentanoic acid) and its soluble salts (e.g., sodium, potassium).

In certain aspects, a bifunctional crosslinker can be added to step (a) to further crosslink the first and second polymeric networks. In one aspect, the crosslinker has two or more acryloyl groups, methacryloyl groups, or a combination thereof. In one aspect, the crosslinker is a polyalkylene oxide glycol diacrylate or dimethacrylate. For example, the polyalkylene can be a polymer of ethylene glycol, propylene glycol, or block co-polymers thereof. In another aspect, the crosslinker is the crosslinker comprises N,N′-methylenebisacrylamide or N,N′-methylenebismethacrylamide. In one aspect, the molar ratio of (meth)acrylic monomer used to the produce the first polymeric network to crosslinker is 100:1 to 20:1. In another aspect, the molar ratio is 100:1, 90:1, 80:1, 70:1, 60:1, 50:1, 40:1, 30:1, or 20:1.

After step (a), the resulting hydrogel is contacted with a solution of multivalent cation, a polycation, or a combination thereof. In one aspect, the hydrogel is immersed in a solution of the multivalent cation and/or a polycation. In one aspect, the solvent is water or a buffered solution typically used in biological applications (e.g., TRIS, TAPS, TAPSO, HEPES, TES, MOPS). The pH of the solution composed of the multivalent cation and/or a polycation can also vary depending upon the number of phosphate groups and the solubility of the multivalent cation and polycation. In one aspect, the pH of the solution of the multivalent cation and polycation is from 6 to 10, 7 to 9, or 7 to 8. Exemplary procedures for incorporating the multivalent cations into the hydrogels are provided in the Examples.

The multivalent cations as used herein have a charge of +2 or greater. In one aspect, the multivalent cation can be a divalent cation composed of one or more alkaline earth metals. For example, the divalent cation can be a mixture of Ca⁺² and Mg⁺². In other aspects, transition metal ions with a charge of +2 or greater can be used as the multivalent cation (e.g., Fe⁺², Fe⁺³, Zn⁺², Al⁺³, Cu⁺², Cu⁺³). In another aspect, the multivalent cation is a rare earth metal such as, for example, lanthanum, terbium, and europium. The counterion of the multivalent cation can vary as well. In one aspect, the counterion is a halide (e.g., chloride), a sulfate, carboxylate, and the like. The type and amount of multivalent cation can modulate the physical properties of the hydrogel.

The polycation is a compound having a plurality of cationic groups at a particular pH. In one aspect, the polycation is a polyamine compound (i.e., a compound possessing two or more amino groups). The amino group can be a primary, secondary, or tertiary amino group that can be protonated to produce a cationic ammonium group at a selected pH.

In one aspect, the polycation is an aminoglycoside antibiotic Aminoglycoside antibiotics are Gram-negative antibacterial therapeutic agents that inhibit protein synthesis and contain as a portion of the molecule an amino-modified glycoside (sugar). Examples of aminoglycoside antibiotics useful herein include streptomycin, tobramycin, kanamycin, gentamicin, neomycin, amikacin, debekacin, sisomycin, netilmicin, neomycin B, neomycin C, neomycin E, or any combination thereof. As will be discussed in detail below, the hydrogels described herein can be used as drug delivery devices.

The design feature of the hydrogels described herein includes two independently cross-linked interpenetrating networks: a soft highly extensible elastic network (i.e., polymeric network) and a stiff brittle sacrificial network formed through non-covalent reversible bonds (i.e., formation of cross-bridges between phosphate groups present in the hydrogel and multivalent cations and/or polycations). Mechanical loading ruptures the non-covalent interactions in the stiff sacrificial network at a critical force and extension corresponding to a pseudo-yield point, which results in strain softening as the elastic network is extended. When unloaded, the elastic network provides a restoring force that guides reformation of the non-covalent bonds, allowing the hydrogel to recover to its initial dimensions and stiffness. The hydrogels described herein can undergo multiple highly hysteretic cycles to repeatedly dissipate strain energy. The Examples demonstrate the unique physical properties of the hydrogels described herein.

As discussed above, the hydrogels described herein can be produced as molded articles. However, in other embodiments, the hydrogels can be processed to produce microgels or nanogels using techniques know in the art. In one aspect, the nanogels or microgels can be produced by inverse emulsion or “mini” emulsion polymerization. In other aspects, larger hydrogels can be mechanically ground into nanogels or microgels. In this embodiment, the microgels or nanogels can be useful in delivering bioactive agents to a subject. The bioactive agents can be any drug including, but not limited to, antibiotics, pain relievers, immune modulators, growth factors, enzyme inhibitors, hormones, mediators, messenger molecules, cell signaling molecules, receptor agonists, or receptor antagonists. For example, when the hydrogels are produced with an aminoglycoside antibiotic, the microgels or nanogels can be administered to a subject that has a bacterial infection. In one embodiment, the microgels or nanogels with aminoglycoside antibiotic can be aerosolized to be administered to a subject having a pulmonary infection.

In certain embodiments, the microgels or nanogels with the bioactive agent can be formulated with one or more multivalent cations in order to modify the release pattern of the bioactive agent from the microgels or nanogels. Additionally, mechanically stressing, e.g., stretching or compressing, the hydrogels can accelerate the release of the bioactive agent. In another embodiment, the microgel or nanogel can have an aminoglycoside antibiotic and Cu⁺² ions as the multivalent cation. In this embodiment, the Cu⁺² ions possess anti-bacterial activity to supplement or enhance the anti-bacterial properties of the aminoglycoside antibiotic. Additionally, the Cu⁺² ions can modulate the release pattern of the aminoglycoside antibiotic.

The microgels or nanogels can be formulated in any excipient the biological system or entity can tolerate to produce pharmaceutical compositions. Examples of such excipients include, but are not limited to, water, aqueous hyaluronic acid, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol. In certain aspects, the pH can be modified depending upon the mode of administration. For example, the pH of the composition is from about 5 to about 8, which is suitable for topical applications. Additionally, the pharmaceutical compositions can include carriers, thickeners, diluents, preservatives, surface active agents and the like in addition to the compounds described herein.

It will be appreciated that the actual preferred amounts of the bioactive agent in the microgels and nanogels in a specified case will vary according to the specific compound being utilized, the particular compositions formulated, the mode of application, and the particular situs and subject being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

The pharmaceutical compositions described herein can be administered in a number of ways depending on whether local or systemic treatment is desired, and on the area to be treated. Administration can be topically (including ophthalmically, vaginally, rectally, intranasally, orally, or directly to the skin). Administration for periodontal disease or gingivitis can be topically via delivery of a gel, paste, or rinse to the diseased gums or periodontal pockets. Formulations for topical administration can include ointments, lotions, creams, gels, drops, suppositories, sprays, liquids and powders. Conventional pharmaceutical carriers, aqueous, powder or oily bases, thickeners and the like can be necessary or desirable. Administration can also be directly into the lung by inhalation of an aerosol or dry micronized powder. Administration can also be by direct injection into the inflamed or degenerating joint space. In other aspects, the hydrogels described herein can be formulated as a coating to be applied to an article that can be implanted in a subject.

Due to the unique properties of the hydrogels described herein, they have numerous applications where it is desirable to use flexible yet strong materials. In one aspect, adhesive hydrogel includes (1) a layer of a hydrogel described herein having a first side and s second side, and (2) an adhesive layer adjacent to the first side of the hydrogel layer, wherein the adhesive comprises (a) a macromer comprising a plurality of phenolic groups covalently bonded to the macromer and (b) an enzyme for catalyzing covalent crosslinking between the phenolic groups in the macromer and phenolic groups present on a substrate, such as a tissue.

The macromer includes a plurality of phenolic groups covalently bonded to the macromer. The number of phenolic groups can vary due to the application of the adhesive hydrogel. In the case when the macromer is a polymer, the phenolic groups can be pendant to the polymer backbone and/or incorporated within the polymer backbone. The number of hydroxyl groups present in each phenolic group can vary as well. In one aspect, each phenolic group has one hydroxyl group. In another aspect, each phenolic group has two or more hydroxyl groups.

In one aspect, the macromer can be composed of one or more synthetic polymers having a plurality of phenolic groups. In one aspect, the macromer is a peptide or protein. For example, the peptide or protein can include one or more tyrosine residues, which have a phenol sidechain.

In another aspect, the macromer can include a polyacrylate having one or more pendant phenolic groups. For example, the macromer can be derived from the polymerization of (meth)acrylic monomers described herein.

In another aspect, the polyanion is a polymer having at least one fragment having the formula V

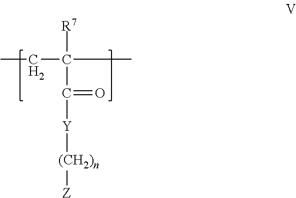

wherein R⁷ is hydrogen or an alkyl group;
n is from 1 to 10;
Y is oxygen, sulfur, or NR⁸, wherein R⁸ is hydrogen, an alkyl group, or an aryl group; and
Z is a phenolic group or a group comprising a phenolic group.

In one aspect, Z is

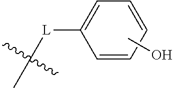

wherein when linker L is not present the phenolic group is directly bonded to the CH₂ group in formula I. In the case when L is present (e.g., a heteroatom such as oxygen or nitrogen or by another organic group), Z is a group comprising a phenolic group.

In one aspect, the phenolic group includes one hydroxyl group. In other aspect, phenolic group can have two hydroxyl groups. For example, the phenolic group includes a dihydroxy-substituted aromatic group capable of undergoing oxidation in the presence of an oxidant. In one aspect, the dihydroxy-substituted aromatic group is an ortho-dihydroxy aromatic group capable of being oxidized to the corresponding quinone. In another aspect, the dihydroxyl-substituted aromatic group is a dihydroxyphenol or halogenated dihydroxyphenol group such as, for example, the catechols (e.g., 3,4 dihydroxyphenol). In the presence of an oxidant such as a peroxide, the dihydroxyl-substituted aromatic group can be oxidized and form new covalent bonds with neighboring groups.

The adhesive layer of the adhesive hydrogel also includes an enzyme for catalyzing covalent crosslinking between the phenolic groups in the macromer and phenolic groups present on a substrate. In one aspect, the enzyme is a peroxidase derived from plant, animal, or bacteria. In another aspect, the peroxidase is a recombinant peroxidase. In a further aspect, the enzyme is horseradish peroxidase. In another aspect, the enzyme is a catechol oxidase.

The combination of the enzyme with the macromer can vary depending upon the selection of the components and the application of the adhesive. In one aspect, the macromer and enzyme are mixed with one another so that the enzyme is physically entrapped within the macromere layer and not covalently attached to the macromer. In other aspects, the enzyme can be covalently bonded to the macromer. For example, horseradish peroxide can be functionalized with activated ester groups for crosslinking to nucleophilic groups on the macromer using techniques known in the art.

In another aspect, the enzyme can be modified with one or more phenolic groups to form covalent bonds with itself to produce a self-crosslinked network within the macromer. In this embodiment, the enzyme can provide a structural component to the adhesive as well enzyme activity.

The enzyme is mixed with the macromer in a manner to ensure the enzyme is evenly distributed throughout the macromer. In certain aspects, depending upon the selection of the macromer, one or more solvents can be used to ensure thorough and stable mixing of the components. Solvents such as, for example, water or an alcohol, can be used particularly if the adhesive layer is to be used in biomedical applications. In one aspect, in order to preserve the activity of the enzyme, the macromere and mixture is lyophilized on the surface of the hydrogel. In this aspect, the enzyme is activated upon hydration of the adhesive layer. In other aspects, enzyme stabilizers can be added to the adhesive layer to prolong the activity of the enzyme. In one aspect, a sugar stabilizer such as, for example, trehalose, can be used herein.

Depending upon the application of the adhesive hydrogel, the adhesive layer can include one or more tackifiers that can be used in combination with the adhesive layer to increase adhesion to a substrate. Depending upon the application, the adhesive can be mixed thoroughly with the tackifier so that the tackifier is dispersed evenly throughout the adhesive layer.

A number of tackifiers known in the art can be used herein. In one aspect, the tackifier is a low modulus hydrophilic polymer such polyacrylic acid or polymethacrylic acid. Other examples of tackifiers include, but are not limited to, acrylics, a butyl rubber, ethylene-vinyl acetate, natural rubber, a nitrile, a silicone rubber, a styrene block copolymer, a vinyl ether, a glycosylated protein, a carbohydrate, or any combination thereof. In the case when the adhesive hydrogel is used in a biomedical application, the pressure sensitive adhesive coating should be biocompatible.

It is also contemplated that the adhesive layer of the adhesive hydrogel can encapsulate one or more bioactive agents. The bioactive agents can be any drug including, but not limited to, antibiotics, pain relievers, immune modulators, growth factors, enzyme inhibitors, hormones, mediators, messenger molecules, cell signaling molecules, receptor agonists, or receptor antagonists.

In another aspect, the bioactive agent can be a nucleic acid. The nucleic acid can be an oligonucleotide, deoxyribonucleic acid (DNA), ribonucleic acid (RNA), or peptide nucleic acid (PNA). The nucleic acid of interest can be nucleic acid from any source, such as a nucleic acid obtained from cells in which it occurs in nature, recombinantly produced nucleic acid, or chemically synthesized nucleic acid. For example, the nucleic acid can be cDNA or genomic DNA or DNA synthesized to have the nucleotide sequence corresponding to that of naturally-occurring DNA. The nucleic acid can also be a mutated or altered form of nucleic acid (e.g., DNA that differs from a naturally occurring DNA by an alteration, deletion, substitution or addition of at least one nucleic acid residue) or nucleic acid that does not occur in nature.

In other aspects, the bioactive agent is used in bone treatment applications. For example, the bioactive agent can be bone morphogenetic proteins (BMPs) or prostaglandins. Bioactive agents known in the art such as, for example, bisphonates, can be delivered locally to the subject.

In another aspect, the adhesive hydrogels described herein further include silver ions entrapped within the adhesive layer and/or deposited on the surface of the adhesive layer. For example, silvers salts such as silver chloride or silver nitrate can be admixed with the macromer and enzyme to entrap the silver salt throughout the adhesive. Alternatively, the silver salt can be applied to the surface of the adhesive layer by spraying or other techniques known in the art. Not wishing to be bound by theory, the enzyme present in the adhesive layer can reduce the silver ions to elemental silver nanoparticles, which possess anti-microbial activity. This is important when the adhesives are used in biomedical applications.

The adhesive can be applied to the surface of the hydrogels described herein by techniques known in the art including spraying or rolling.

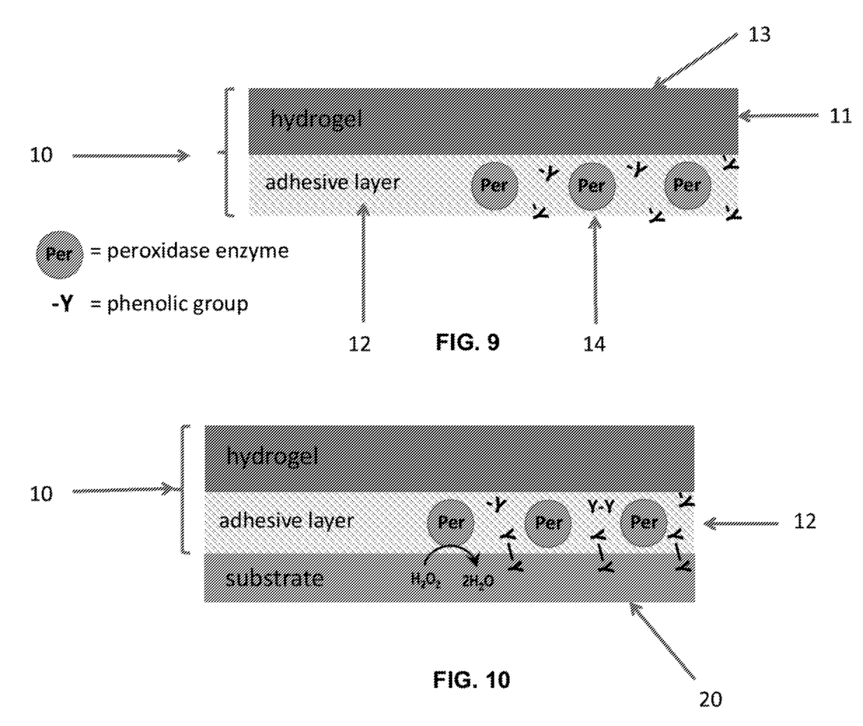

An exemplary feature of the adhesive hydrogel is provided in FIG. 9. Referring to FIG. 9, the adhesive hydrogel 10 is composed of the hydrogel 11 and adhesive layer 12, where the adhesive layer contains a peroxidase enzyme mixed with a macromer having a plurality of phenolic groups.

In certain aspects, the adhesive hydrogel includes a backing on the second surface of the hydrogel. Referring to FIG. 9, the backing can be applied to the surface 13 of the hydrogel 11. In this embodiment, the hydrogel is sandwiched between the adhesive layer and the backing.

The material of the backing can vary depending upon the application of the adhesive hydrogel. In one aspect, the backing is composed of a non-degradable material. In other aspects, the backing is composed of a biodegradable material. In other aspects, the backing is composed of a biocompatible material. The backing can range from stiff or rigid materials to resilient materials to viscoelastic materials. In one aspect, the backing is a water insoluble sheet or film (e.g., silicone, polyurethane, polyfluoropolymers such as PTFE and expanded PTFE), a woven fabric (e.g., a polyester such as Dacron), a degradable film (e.g., polycaprolactone), a regenerated cellulose sheet, a decellularized tissue scaffold (e.g., human amniotic membranes, bovine pericardium, porcine mucosa), a metal plate or foil (e.g., titanium or stainless steel.

In certain aspects, it may be desirable for the adhesive hydrogel to have adhesive layers on both sides of the hydrogel. In this embodiment, the hydrogel is sandwiched between two layers of adhesive coating. In other embodiments, a removable, protective layer can be applied to the surface of the adhesive coating. Protective layers known in the art can be used in this embodiment.

The adhesive hydrogels described herein can be adhered to a wet surface without the need for drying the surface. The adhesive hydrogels are particularly useful in biomedical applications, in aqueous physiological conditions. When applied to the surface of a substrate of interest, the adhesive layer on the adhesive hydrogels can form covalent bonds with the substrate to produce a bond between the substrate and the adhesive layer. Not wishing to be bound by theory, when the adhesive layer of the adhesive hydrogels is in contact with a substrate surface possessing a plurality of phenolic groups, the enzyme in the adhesive layer in the presence of peroxide source, catalyzes crosslinking between phenolic groups in the adhesive layer and the substrate. This mechanism is depicted FIG. 10, where new covalent bonds (Y-Y) are formed between the adhesive layer 12 and substrate 20.

In one aspect, the peroxide source is a peroxide compound such as, for example, hydrogen peroxide (H₂O₂). In other aspect, the peroxide source is a compound that produces hydrogen peroxide in situ. In one aspect, when the substrate is a tissue in a subject (e.g., bone, muscle, cartilage, ligaments, tendons, soft tissues, organs, or skin), superoxide dismutase (SOD) or glucose oxidase (i.e., peroxide sources) present in the wound can generate hydrogen peroxide in situ. Therefore, in this aspect, the substrate does not need to be contacted with an additional peroxide source prior to application of the adhesive hydrogel. However, in the situation when the peroxide source is not produced in situ, the substrate can be contacted with the peroxide source prior to application of the adhesive hydrogel. For example, a peroxide source such as superoxide dismutase (SOD) or glucose oxidase can be incorporated in the adhesive layer of the adhesive hydrogel.

In other aspects, in order to enhance the adhesion between the adhesive layer and the substrate, the surface of the substrate can be primed with a layer of adhesive described herein having a plurality of phenolic groups. In this aspect, the adhesive applied to the surface of the substrate can be the same or different than the adhesive on the adhesive hydrogel.

In one aspect, the adhesive hydrogels described herein can be used to repair a number of different bone fractures and breaks. Examples of such breaks include a complete fracture, an incomplete fracture, a linear fracture, a transverse fracture, an oblique fracture, a compression fracture, a spiral fracture, a comminuted fracture, a compacted fracture, or an open fracture. In one aspect, the fracture is an intra-articular fracture or a craniofacial bone fracture. Fractures such as intra-articular fractures are bony injuries that extend into and fragment the cartilage surface. The adhesive hydrogels may aid in the maintenance of the reduction of such fractures, allow less invasive surgery, reduce operating room time, reduce costs, and provide a better outcome by reducing the risk of post-traumatic arthritis. In other aspects, the adhesive hydrogels can be used to join small fragments of highly comminuted fractures. In this aspect, small pieces of fractured bone can be adhered to an existing bone.

In other aspects, the adhesive hydrogels can be used as a patch to bone and other tissues such as, for example, cartilage, ligaments, tendons, soft tissues, organs, and synthetic derivatives of these materials. In one aspect, the patch can be a tissue scaffold or other synthetic materials or substrates typically used in wound healing applications. The adhesive hydrogels can be used to position biological scaffolds in a subject. In certain aspects, the scaffold can contain one or more drugs that facilitate growth or repair of the bone and tissue. In other aspects, the scaffold can include drugs that prevent infection such as, for example, antibiotics. For example, the scaffold can be coated with the drug or, in the alternative, the drug can be incorporated within the scaffold so that the drug elutes from the scaffold over time.

In other aspects, the adhesive hydrogels can adhere a substrate to bone. For example, implants made from titanium oxide, stainless steel, or other metals are commonly used to repair fractured bones. In one aspect, the adhesive hydrogel composed of adhesive on either side can be applied to the metal substrate and the bone to adhere the substrate to the bone. In other aspects, the substrate can be a fabric (e.g., an internal bandage), a tissue graft, or a wound healing material. Thus, in addition to bonding bone fragments, the adhesive hydrogels can facilitate the bonding of substrates to bone, which can facilitate bone repair and recovery.

The adhesive hydrogels can be used in a variety of other surgical procedures. For example, the adhesive hydrogel can be applied as a covering to a wound created by the surgical procedure to promote wound healing and prevent infection. In one aspect, the adhesive hydrogels can be used to treat ocular wounds caused by trauma or by the surgical procedures. In one aspect, the adhesive hydrogels can be used to repair a corneal or schleral laceration in a subject. In other aspects, the adhesive hydrogels can be used to facilitate healing of ocular tissue damaged from a surgical procedure (e.g., glaucoma surgery or a corneal transplant).

In another aspect, the adhesive hydrogels can be used to seal a fistula in a subject. A fistula is an abnormal connection between an organ, vessel, or intestine and another structure such as, for example, skin. Fistulas are usually caused by injury or surgery, but they can also result from an infection or inflammation. Fistulas are generally a disease condition, but they may be surgically created for therapeutic reasons. In other aspects, the adhesive hydrogels can prevent or reduce undesirable adhesion between two tissues in a subject, where the method involves contacting at least one surface of the tissue with the adhesive hydrogel.

In certain aspects, the adhesive hydrogel possesses bioactive properties. In other aspects, the adhesive layer contains silver nanoparticles, where the particles can also behave as an anti-bacterial agent. The rate of release can be controlled by the selection of the materials used to prepare the complex as well as the charge of the bioactive agent if the agent is a salt. Thus, in this aspect, the adhesive hydrogel can perform as a localized controlled drug release depot. It may be possible to simultaneously fix tissue and bones as well as deliver bioactive agents to provide greater patient comfort, accelerate bone healing, and/or prevent infections.

As discussed above, the hydrogel described herein can include bioactive agents that can be tuned for desired release patterns. In one embodiment, the hydrogel can include an aminoglycoside antibiotic as the polycation, where the adhesive hydrogel is an anti-bacterial agent.

In addition to biomedical applications, the adhesive hydrogels can be used in a number of non-medical applications that contain water or that will be exposed to an aqueous environment. For example, the adhesive hydrogels can be applied to an underwater substrate that is cracked in order to seal the crack. For example the adhesive hydrogels can be constructed with the appropriate backing and adhesive to seal cracks in boat hulls.

The adhesive hydrogels produced herein can be stored on the shelf until ready for use. In situations where a peroxide source is needed, a kit composed of the adhesive hydrogel and a container of peroxide source can be used when needed. The kit can include additional components such a primer composed of a macromer described herein.

### Examples

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials

Phosphorus(V) oxychloride, 2-hydroxyethyl methacrylate, triethylamine, and glycidyl methacrylate were purchased from Alfa Aesar (Ward Hill, Mass.). 4-Methoxyphenol was purchased from Tokyo Chemical Industry Co., Ltd, (Tokyo, Japan). Methacrylic acid, 2,2′-azobis(2-methylpropionitrile), acrylamide, N,N0-methylenebisacrylamide, and N,N,N′,N′-tetramethylethylenediamine were purchased from Sigma Aldrich (St Louis, Mo.) Ammonium persulfate was purchased from Fischer Scientific (Pittsburgh, Pa.).

### Phosphate Monomer Synthesis

2-(Methacryloyloxy)ethyl phosphate (MOEP) was synthesized as follows. Phosphorus oxychloride (33.9 g, 220 mmol) was mixed with hydroxyl-ethyl-methacrylamide (HEMA) at a 0.7:1 molar ratio in dry toluene (480 ml) under flowing argon. The reaction was stirred at 4° C. while triethylamine (TEA) (77 ml) was added slowly over 10 min Following addition of TEA, the reaction was stirred under argon gas for 6 h at 22° C., then filtered to remove precipitated salt. The reaction was cooled to 4° C. before addition of DI water (480 ml), then stirred under argon at 22° C. for 15 h. The reaction was extracted twice with diethyl ether (100 ml). The organic layer was discarded. The aqueous layer was extracted using tetrahydrofuran (THF) and diethyl ether (1:2, 12×225 ml), then dried over anhydrous sodium sulfate. The monomer was verified by ¹H and ³¹P NMR.

Synthesis of polyMOEP-MA

PolyMOEP was synthesized by free radical polymerization of MOEP (85 mol %), and methacrylic acid (15 mol %) in methanol (12.5 ml mg⁻1 MOEP). The reaction was initiated with azo-bisisobutyronitrile (AIBN, 4.5 mol %) at 55° C., and proceeded for 15 h. The product was precipitated with acetone, then dissolved in water (200 ml H₂O per 17 g pMOEP). Subsequently, methacrylate groups (MA) were grafted onto the methacrylic acid sidechains with glycidyl methacrylate in 9-fold molar excess relative to the methacrylate sidechains. The methacrylated pMOEP (pMOEP-MA) was purified by tangential flow filtration using a Millipore Pellicon 3 cassette filter with an Ultracel 10 kD membrane. The polymer was washed with 10 volumes of water during filtration. The pH was adjusted to 7.3 with NaOH, the product lyophilized, and stored at −20° C. The resulting phosphate prepolymer contained 62.6 mol % phosphate sidechains, 10.9 mol % hydroxy ethylmethacrylate (HEMA), and 26.5 mol % MA sidechains, as determined by ¹H and ³¹P NMR, where the source of HEMA is from partial hydrolysis of the phosphate groups of MOEP during copolymerization. The molecular mass (Mm) and polydispersity index (PDI) of pMOEP-MA was determined by size exclusion chromatography (SEC) using an Amersham Pharmacia AKTAFPLC system equipped with Wyatt MiniDawn Treos (light scattering) and Wyatt Optilab rEX (refractive index) detectors. The Superose 6 HR 10/30 column was equilibrated with 0.1 M sodium acetate (pH 6.5) containing 30% (vol/vol) acetonitrile. The average Mm and PDI were calculated using Wyatt MiniDawn ASTRA software to be 89 kg mol⁻1 and 2.6, respectively.

### Hydrogel Polymerization

Hydrogels were formed by free radical polymerization of acrylamide (Aam) and N,N′-methylenebisacrylamide (bis-Aam) with the pMOEP-MA prepolymer in 150 mM NaCl and 5 mM tris (pH 8.0) (FIG. 1). The total wt % of Aam, bis-Aam and MOEP-MA pre-polymer was held constant at 7.5 wt/vol %, while the amount of the prepolymer was varied from 0.5% wt/vol % to 7.0 wt/vol %. The molar ratio of Aam to bis-Aam was 60:1. Polymerization was initiated by adding 10% ammonium persulfate (APS) and tetramethylethylenediamine (TEMED) to final concentrations of 70 mg mil and 2.4 ml mil, respectively, to the monomer/pre-polymer solution. Polymerization proceeded in dog bone-shaped molds for 90 min at 22° C. Molds were laser cut from 2 mm thick silicone rubber sheets, which were clamped between two acrylic plates to form the complete molds. A layer of mineral oil was floated on top of the polymerization reaction to limit exposure to oxygen. Polymerized gels were soaked in 150 mM NaCl, 5 mM tris (pH 8) with repeated changes of solution for 24 h to remove unreacted materials.

### Hydrogel Metal Ion Exchange

Hydrogels were immersed in 150 mM NaCl, 5 mM tris (pH 8.0) with metal ions (Ca²⁺, Mg²⁺, or Zn²⁺) added in 5 mM increments up to 50 mM over 24 h.

Gradual addition of metal ions improved the homogeneity of the deswelled hydrogels. The hydrogels were then soaked in 50 mM metal ion and 5 mM tris (pH 8.0) for an additional 24 h with frequent solution changes. Images of hydrogels were recorded using a dissection microscope during volume equilibration and their dimensions were measured using ImageJ. Isotropic shrinking was assumed to calculate volume changes. Hydrogels were considered to be fully equilibrated when the volume reached steady state. Hydrogel density was measured by the buoyancy method using an analytical balance density kit (Mettler Toledo, Inc.) and calculated using the equation:

$${ρ}_{sample} = \frac{( sample {weight}_{air} ) \times ( {ρ}_{water} - {ρ}_{air} )}{( sample {weight}_{air} - sample {weight}_{water} )} + {ρ}_{air}$$

The density of water was corrected for temperature. Metal phosphate ratios were determined by ICP-OES of two independent hydrogel specimens at a commercial testing facility (Advanced Labs, Salt Lake City, Utah).

### Mechanical Testing of Hydrogels

Hydrogels were strained while submerged in 5 mM tris, pH 8.0, containing 5 mM of the test metal ion on an Instron 3342 material test system controlled with Bluehill software (Instron, Inc.). Ca²⁺-equilibrated hydrogels were strained at rates ranging from 0.01 to 1.0 s⁻1. Strain to fracture and cyclical strain tests were done at 0.15 s⁻1.

### Infrared Spectroscopy

Sodium equilibrated hydrogels were incubated overnight in 10 mM Na+ EDTA to remove rouge divalent metal ions potentially scavenged during polymerization and processing. Na⁺ gels were stored in 1 mM EDTA to prevent binding of trace divalent metal ions. Divalent metal ion hydrogels were equilibrated with the respective metal ion as described above. After volume equilibration, the samples were rinsed with water, then lyophilized to remove water, and crushed into a powder using an agar mortar and pestle before applying to the diamond ATR crystal. The IR spectra were normalized to the intensity of an absorption band centered at 1665 cm⁻1, which corresponds to absorption by amide groups in the polymethacrylamide backbone. A linear baseline correction was applied to the intensity normalized spectra between 800 and 1300 cm⁻1, which contains several phosphate vibrational modes. ATR-FTIR absorbance spectra were collected using a Nicolet 6700 spectrometer (Thermo Scientific, FL) with a diamond Smart iTR accessory, a deuterated triglycine sulfate detector, and a KBr/Ge mid-infrared optimized beamsplitter. Spectra were recorded with a resolution of 4 cm⁻1 and as 512 averaged scans.

### Processing of Experimental Data

Data was processed in matlab (MathWorks). Linear fits to the initial part of the stress strain curve were used to estimate the initial modulus. The yield point was determined using a 5% strain offset from the initial linear portion of the curve. Energy dissipation, strain cycle hysteresis, was computed by subtracting the trapezoidal integration of the reverse curve from the forward curve of cyclical tests. Residual strain was measured by extending the initial linear portion of the stress strain curve (disregarding toe regions) through the base line.

### Synthesis of Divalent Metal-Ion Crosslinked Hydrogels

Polymethacrylate random copolymers were synthesized with varying mol % of ethylphosphate (MOEP), ethyl-hydroxy (HEMA) sidechains, and carboxylate (MAA) sidechains (FIG. 1). The MAA groups were subsequently grafted with glycidyl methacrylate as crosslinking groups. To prepare the hydrogels, the sodium salt of methacrylated polyphosphate (pMOEP-MA) prepolymers were mixed with acrylamide (AAM) and bisacrylamide (bis-AAM) monomers and copolymerized in 150 mM NaCl, and 5 mM tris (pH 8.0). The total wt/vol % of polymer in the hydrogels was kept constant at 7.5 wt/vol %. During polymerization, the pMOEP-MA prepolymer became crosslinked into the pAAM network through the MA sidechains (FIG. 1). The resulting dog bone-shaped hydrogels, with Na⁺ counterions, were clear and transparent.

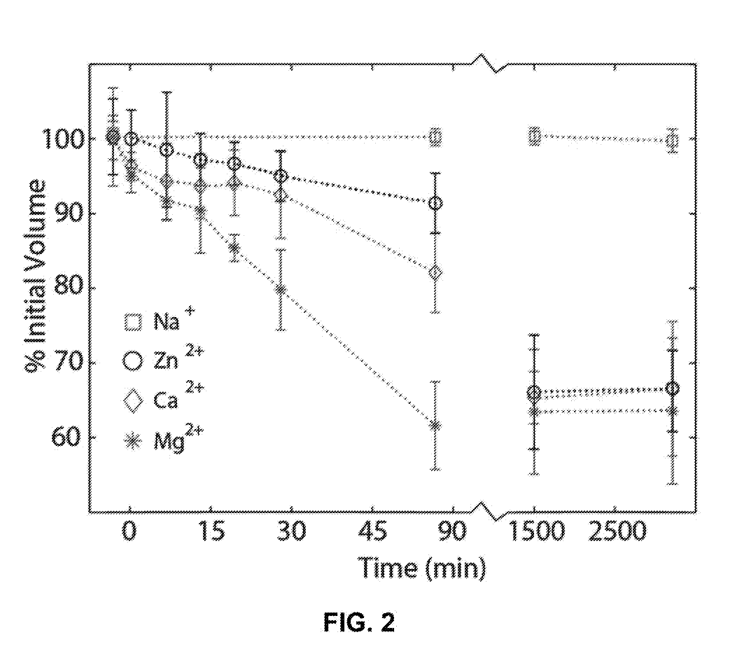

As Na⁺ was exchanged with the divalent metal-ions, Mg²⁺, Ca²⁺, and Zn²⁺, the hydrogels shrank to about 65% of their initial volume (Table 1). The final volume had little dependence on the divalent metal ion species (FIG. 2). However, the hydrogels shrank fastest in Mg²⁺, equilibrating in 90 min, whereas volume equilibration in both Ca²⁺ and Zn²⁺ took approximately 24 h. During divalent metal ion exchange, the initially transparent Nathydrogels became slightly translucent. The resulting divalent ion-equilibrated DN hydrogels had three types of crosslinks within and between networks: covalent bis-AAM junctions between pAAM chains, covalent bis-AAM junctions between pAAM and methacrylated side chains in pMOEP networks, and reversible phosphate/metal ion junctions within the pMOEP network, which were likely a mix of inter- and intramolecular crosslinks (FIG. 1).

TABLE 1

| Ion | Zn²⁺ | Ca²⁺ | Mg²⁺ | Na⁺ |
|---|---|---|---|---|
| Volume (% of initial) | 66.2 ± 1.2 | 66.5 ± 2.1 | 63.6 ± 3.3 | 97.2 ± 2.4 |
| Water (wt %) | 53.6 ± 1.3 | 55.6 ± 2.1 | 56.3 ± 1.9 | 92.5 ± 0.5 |
| Density (g cm_3) | 1.10 ± 0.02 | 1.07 ± 0.02 | 1.05 ± 0.02 | 1.01 ± 0.01 |
| M/P molar ratio | 3.9 | 1.7 | 1.5 | — |
| Initial modulus (MPa) | 34.2 ± 2.5 | 10.3 ± 3.5 | 0.1 ± 0.04 | 0.04 ± 0.01 |
| Yield stress (MPa) | 3.5 ± 0.4 | 1.8 ± 0.2 | No yield | No yield |
| Stress at fracture (MPa) | 3.8 ± 0.3 | 1.9 ± 0.1 | 0.3 ± 0.01 | 0.05 ± 0.004 |
| Elongation at fracture (%) | 40 ± 10 | 90 ± 50 | 220 ± 20 | 227 ± 14 |
| Work to fracture (MJ m⁻³) | 10.4 ± 0.4 | 10.5 ± 1.2 | 0.3 ± 0.004 | 0.09 ± 0.01 |

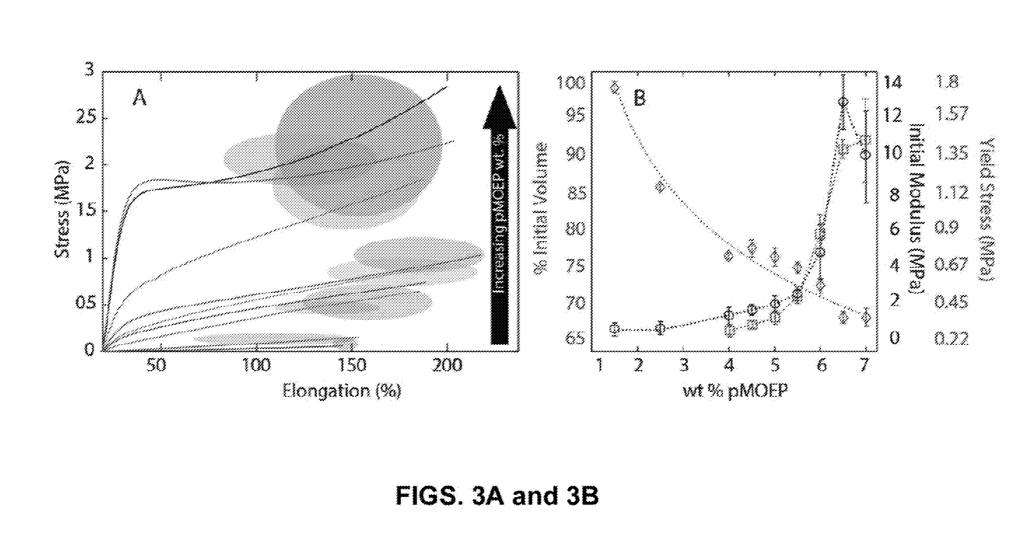

The mechanical effect of varying the ratio of the pMOEP-MA prepolymer network to the pAAM network in hydrogels equilibrated with Ca²⁺ ions was evaluated by tensile testing. The concentration of pMOEP-MA prepolymer was varied from 1.5 to 7.0 wt/vol % while holding the total polymer/monomer concentration constant at 7.5 wt/vol % (FIG. 3A). The hydrogels were strained to failure at room temperature (20-22° C.) while fully submerged in a water bath to prevent water evaporation and to limit potential effects of uneven water flux out of and into the gels. The bath solutions contained 5 mM Ca²⁺ and were buffered at pH 8.0, above the pKₐ₂ of the phosphate sidechains. At the lowest ratio of pMOEP-MA to pAAM, 1.5:6.0 wt/vol %, the Ca²⁺-equilibrated hydrogels were soft with an initial modulus of 0.020±0.004 MPa. The stress increased linearly with strain until fracture occurred at 0.054±0.002 MPa and less than 150% strain (FIG. 3A). As the pMOEP-MA to pAAM ratio was increased to above 5 wt/vol % pMOEP-MA, the initial modulus rose sharply, strain at fracture increased toward 200%, and yield-like behavior—dramatic strain softening—appeared around 20% elongation (FIG. 3A). Hydrogel toughness, as reflected in the work of extension to fracture (FIG. 3B), also increased sharply with increasing pMOEP-MA, due primarily to the increase in yield stress of the hydrogels.

Hydrogel synthesis using pMOEP-MA as a prepolymer with a high mol % of phosphate sidechains resulted in toughened Ca²⁺-crosslinked hydrogels. Other hydrogel synthesis methods failed to produce toughened hydrogels. For example, hydrogels of 7.5 wt/vol % pMOEP-MA with no pAAM, were brittle and frequently fractured during equilibration with divalent metal ions. Hydrogels prepared with 6.5 mol % pMOEP-MA with only 40 mol % phosphate sidechains stiffened considerably with Ca²⁺, but did not display yield-like behavior, shrank less during equilibration with Ca²⁺, and were less tough (not shown). Hence, further hydrogel mechanical characterization was done with hydrogels synthesized with 6.5 wt/vol % pMOEP-MA and 1.0 wt/vol % pAAM/bis-AAM.

### Hysteresis and Self-Recovery Kinetics of Ca²⁺-Crosslinked Hydrogels During Cyclical Loading

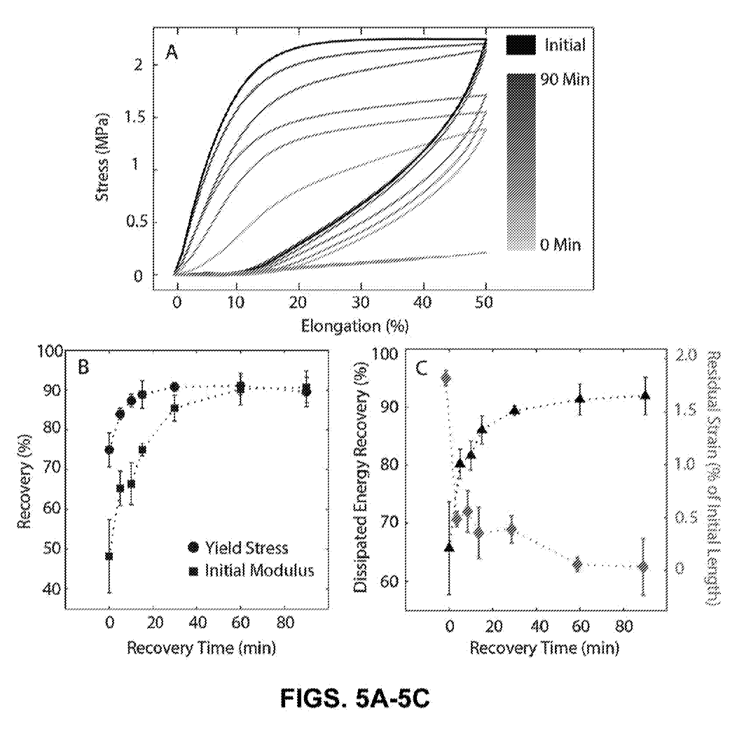

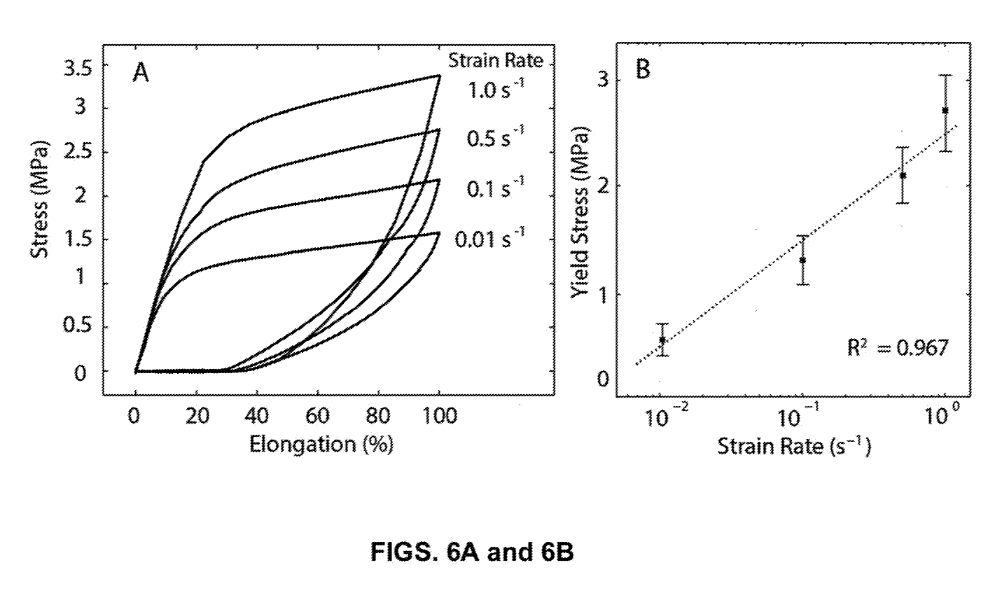

The yield-like response of Ca²⁺ hydrogels was not a permanent plastic deformation. Instead, the initial length, modulus, and yield stress of hydrogels strained to 50% recover approximately 90% of their initial values within 90 min after unloading (FIGS. 5 and 6). Hence, we refer to the phenomenon as pseudo-yield. The area within the forward and reverse curves of the highly hysteretic cycles represents dissipated strain energy, which also recovered to approximately 90% of the initial cycle value within 90 min. The recovery did not fit a single exponential process. In contrast, Mg²⁺ hydrogels had a linear elastic response to cyclical strains, displaying little hysteresis (FIG. 5A, green curves). Hydrogels equilibrated with Zn²⁺ were more brittle beyond the pseudo-yield point and could not be reliably strained to 50% elongation. Therefore the rate of refolding was not determined.

### Strain Rate Dependence of Ca²⁺-Crosslinked Hydrogels

The pseudo-yield stress of Ca²⁺-equilibrated hydrogels strained to 100% at strain rates ranging over three orders of magnitude increased 5-fold (FIG. 6B). Likewise, the initial modulus, work of extension, and dissipated energy increased by, 60%, 2-fold, and 2.3-fold, respectively (not shown). Pseudo-yield stress had a logarithmic dependence on strain rate (FIG. 6B). Strain rate had little effect on residual strain, which varied by only 5% over the range of strain rates.

### Metal Ion Species Dependence of Hydrogel Toughness

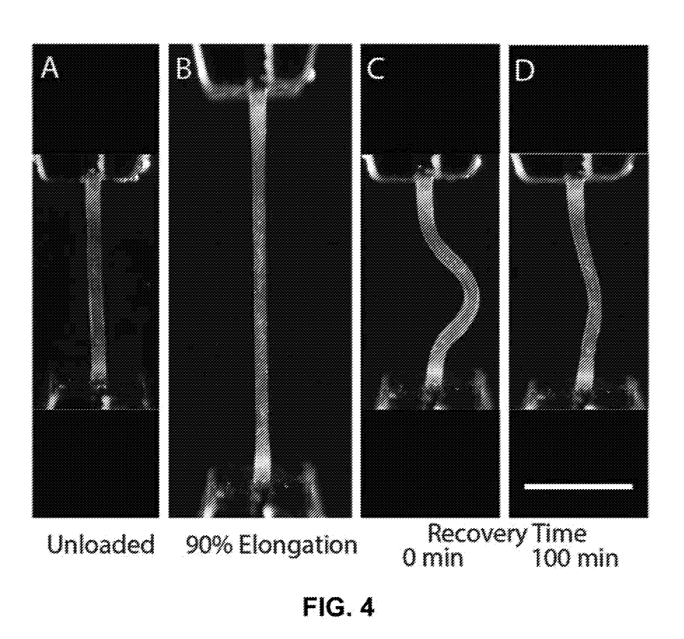

Hydrogels containing Na⁺ counter ions were soft, linear elastomers that could be elongated about 250% before fracture (FIG. 4 and Table 1). Exchange with divalent metal ions increased the pseudo-yield stress in the following order: Mg²⁺<Ca²⁺<Zn²⁺. Hydrogels exchanged with Mg²⁺, like Na⁺ hydrogels, were soft and displayed a linear dependence of stress on strain, whereas Ca²⁺ and Zn²⁺ hydrogels both displayed dramatic strain softening (yield-like) behavior around 20% strain. Although Zn²⁺ hydrogels fractured soon after the yield point, at strains of 40% compared to average strains of 90% for Ca²⁺ hydrogels, the work to fracture of Ca²⁺ and Zn²⁺ was nearly the same, 10.4 and 10.5 MJ m⁻3, respectively, more than three times higher than Mg²⁺ (Table 1).

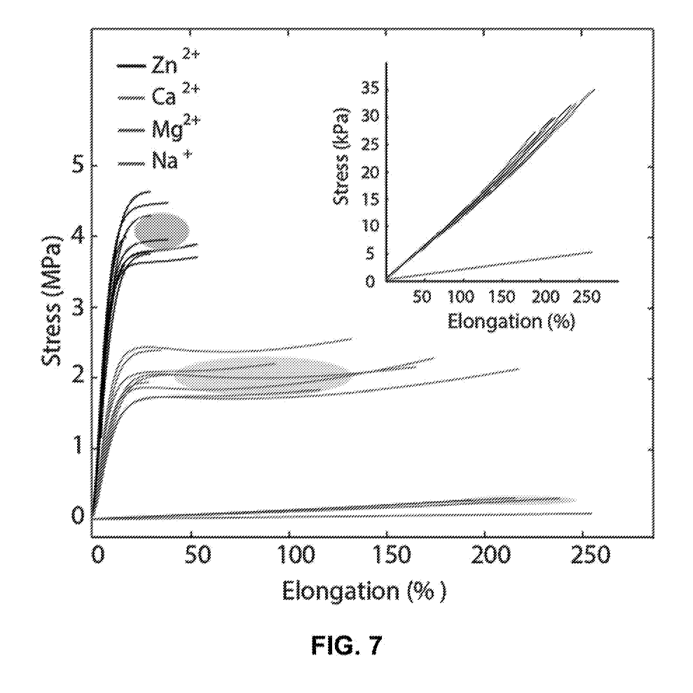

Above a threshold concentration of phosphate sidechains on the pMOEP prepolymer, exchange of monovalent Na⁺ with divalent metal ions resulted in collapse of the hydrogel structure, accompanied by exclusion of about 40% of its equilibrium water mass (Table 1), and a change in appearance from transparent to slightly translucent. Mechanically, the hydrogels transitioned from soft and elastic to tough and viscoelastic with non-permanent strain softening (yield) at a critical stress (FIG. 7).

### IR Spectroscopy of Divalent Metal-Ion Crosslinked Hydrogels

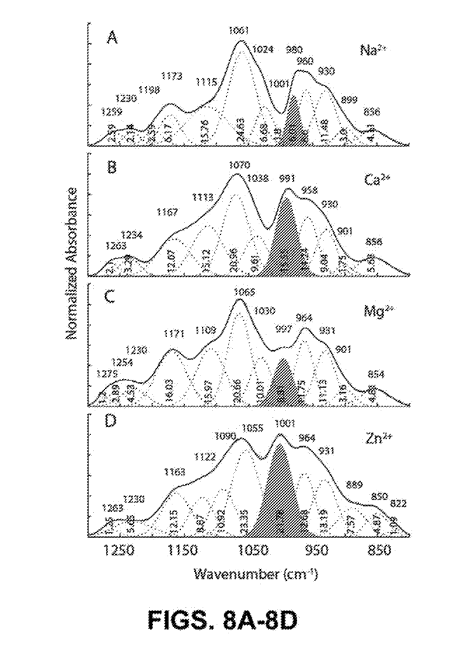

Interactions of divalent metal ions with phosphate sidechains was evaluated by IR spectroscopy (FIG. 8). Bands corresponding to degenerate P—O⁻ symmetric stretching modes occur between 950 and 1050 cm⁻1. The Na⁺ absorption band centered at 980 cm⁻1 corresponds to the combined absorption of two P—O⁻ bonds of dibasic phosphate. The Na⁺ absorption band centered at 980 cm⁻1 corresponds to the combined absorption of two P—O⁻ bonds of dibasic phosphate. The 962 cm⁻1 band is not due to a phosphate vibration based on pH titrations (not shown). The 980 cm⁻1 was blue-shifted B11, 17, and 21 cm⁻1 for Ca²⁺, Mg²⁺, and Zn²⁺, respectively. The absorbance intensity of the shifted band increased in the order: Zn²⁺>Ca²⁺>Mg²⁺.

### Hydrogel Deswelling During Tobramycin Loading

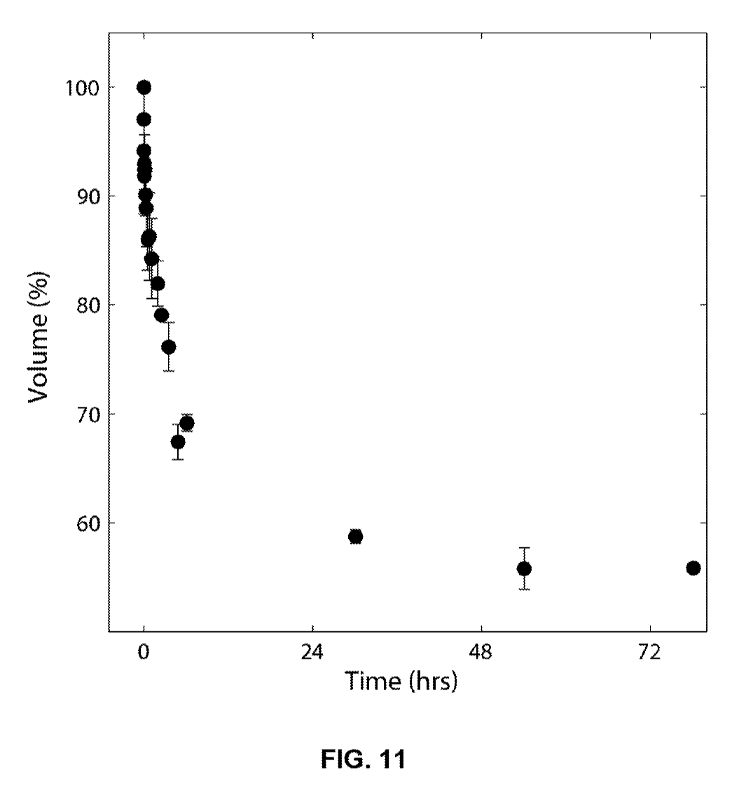

Three hydrogels (6.5 wt/vol % pMOEP, 1 wt/vol % pAAm) were immersed in 5 ml of 5 mM Tobramycin, 150 mM NaCl at pH 12 and photographed for 2 hours. The solution was then adjusted to pH 7.5 and the hydrogels were imaged for 72 hrs. Volume changes were measured from the images using ImageJ. (FIG. 11). Hydrogels were considered fully equilibrated when the volume reached steady state.

Tobramycin Release Kinetics from Polyphosphate Hydrogels.

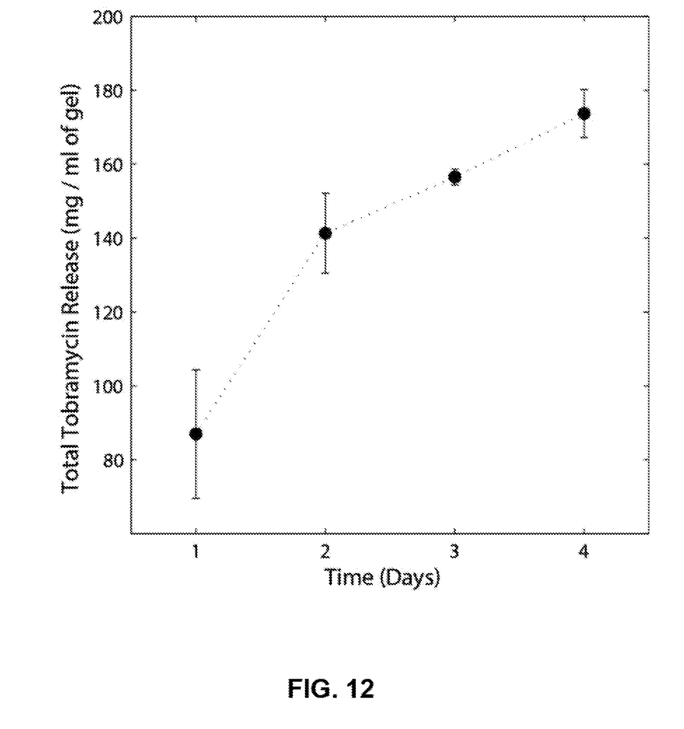

Three hydrogels (6.5 wt/vol % pMOEP, 1 wt/vol % pAAm) were incubated in 5 ml of 10 mM Tobramycin in a 150 mM NaCl at pH 7.5 for 24 hours. The loading solution was replaced with 5 ml of balanced salt solution (BSS) pH 7.5 containing 0.64% NaCl, 0.075% KCl, 0.048% CaCl₂, 0.03% MgCl₂. The BSS solution was replaced every 24 hrs and the amount of the tobramycin released from the hydrogels into the solution was determined using ninhydrin. The cumulative release, mg per ml of the hydrogel, over four days is shown in FIG. 12.

Not wishing to be bound by theory, the divalent cations crosslinked the polyphosphate prepolymer network, both intra and intermolecularly, through the phosphate sidechains into dense partially dehydrated clusters, as illustrated in FIG. 1, that function as pseudo-domains. The collapsed phosphate prepolymer clusters are connected to one another through the elastic polyacrylamide network. The toughening effect—the extra work required to fracture the Ca²⁺ equilibrated hydrogels versus the Na⁺ equilibrated hydrogels—was due to energy absorbed and dissipated by rupture and unfolding of the Ca²⁺ phosphate crosslinked clusters. The dense clusters functioned as a series of sacrificial yield domains undergoing sequential, viscous unfolding and extension in the stress plateau region. Rupture of the Ca²⁺ phosphate crosslinked clusters was reversible, which allowed the domain-like regions to slowly reform when unloaded, guided by the memory of the elastic polyacrylamide network. About 90% of the capacity to dissipate strain energy at moderate strain rates was recovered within 90 min. The less than complete recovery suggested some permanent damage occurred during the first strain cycle.

The stress response of the hydrogels can be tuned to some extent by multivalent metal ion selection, as one means to design hydrogels to meet the specifications of a particular application. The greater stiffness and strength of the Ca2+ and Zn2+ hydrogels (Table 1) may be due to a greater propensity for their hydration shells to be displaced by inner sphere phosphate oxygen bonds, which may result in effectively stronger, load bearing, inter- and intra-chain crosslinks.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed:

1. A hydrogel comprising (a) a first polymeric network comprising a polymer derived from a (meth)acrylic monomer; (b) a second polymeric network comprising a polyphosphate, wherein the first polymeric network and second polymeric network are covalently crosslinked with each other, and (c) a plurality of multivalent cations, a polycation, or a combination thereof that non-covalently crosslinks the second polymeric network.

2. A hydrogel produced by the process comprising
a. polymerizing a (meth)acrylic monomer to produce a first polymeric network in the presence of (1) a second polymeric network comprising a polyphosphate prepolymer comprising a plurality of phosphate groups and a plurality pendant acryloyl groups, pendant methacryloyl groups, or a combination thereof, and (2) a free radical initiator, wherein the first polymeric network and the second polymeric network are covalently crosslinked with each other to produce a first hydrogel; and
b. contacting the first hydrogel with a multivalent cation, a polycation, or a combination thereof to non-covalently crosslink the first second polymeric network.

3. The hydrogel of claim 1, wherein the polycation comprises an aminoglycoside antibiotic.

4. The hydrogel of claim 2, wherein the polycation comprises an aminoglycoside antibiotic.

5. The hydrogel of claim 3, wherein the aminoglycoside antibiotic is streptomycin, tobramycin, kanamycin, gentamicin, neomycin, amikacin, debekacin, sisomycin, netilmicin, neomycin B, neomycin C, neomycin E, or any combination thereof.

6. The hydrogel of claim 4, wherein the aminoglycoside antibiotic is streptomycin, tobramycin, kanamycin, gentamicin, neomycin, amikacin, debekacin, sisomycin, netilmicin, neomycin B, neomycin C, neomycin E, or any combination thereof.

7. The hydrogel of claim 2, wherein the polyphosphate prepolymer has a molecular weight of 1,000 Da to 200,000 Da.

8. The hydrogel of claim 2, wherein the polyphosphate prepolymer is produced by (1) polymerizing a or a phosphate (meth)acrylic monomer with one or more (meth)acrylic monomers to produce a first polymer, and (2) grafting acryloyl groups, methacryloyl groups, or a combination thereof to the first polymer.

9. The hydrogel of claim 8, wherein the phosphate (meth)acrylic monomer has the formula I 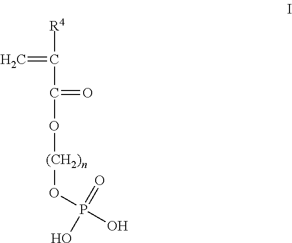
wherein R⁴ is hydrogen or an alkyl group, and n is from 1 to 10.

10. The hydrogel of claim 9, wherein R⁴ is methyl and n is 2.

11. The hydrogel of claim 9, wherein the phosphate (meth)acrylic monomer of formula I is polymerized with acrylic acid, methacrylic acid, a hydroxyalkyl methacrylate, a hydroxyalkyl acrylate, a hydroxyl-substituted (lower alkyl)acrylate, a hydroxylalkyl acrylamide, a hydroxylalkyl methacrylamide, a hydroxyl-substituted (lower alkyl)methacrylate acrylamide, methacrylamide, a (lower alkyl)acrylamide, a (lower alkyl)methacrylamide, a hydroxyl-substituted (lower alkyl)acrylamide, a hydroxyl-substituted (lower alkyl)methacrylamide, or any combination thereof.

12. The hydrogel of claim 9, wherein the phosphate (meth)acrylic monomer of formula I is polymerized with acrylic acid or methacrylic acid.

13. The hydrogel of claim 2, wherein the polyphosphate prepolymer comprises a random copolymer comprising the units in formula III 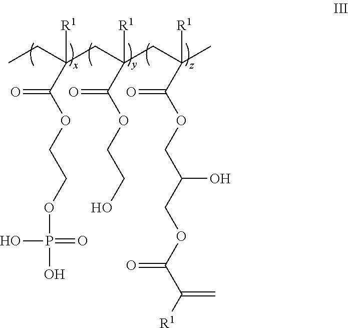
wherein x is from 40 to 90 mol %, y is from 1 to 30 mol %; and z is from 1 to 30 mol % of the polyphosphate prepolymer; and
each R¹ is independently hydrogen or methyl.

14. The hydrogel of claim 13, wherein x is from 50 to 70 mol %; y is from 5 to 20 mol %; and z is from 20 to 30 mol % of the polyphosphate prepolymer, and each R¹ is methyl, and the molecular weight is 1,000 Da to 100,000 Da.

15. The hydrogel of claim 2, wherein the (meth)acrylic monomer comprises acrylic acid, methacrylic acid, hydroxyalkyl methacrylate, a hydroxyalkyl acrylate, acrylamide, methacrylamide, a (lower alkyl)acrylamide, a (lower alkyl)methacrylamide, a hydroxyl-substituted (lower alkyl)acrylamide, a hydroxyl-substituted (lower alkyl)methacrylamide, or any combination thereof.

16. The hydrogel of claim 2, wherein the (meth)acrylic monomer comprises acrylamide or methacrylamide.

17. The hydrogel of claim 2, further comprising in step (a) a crosslinker comprising two or more acryloyl groups, methacryloyl groups, or a combination thereof.

18. The hydrogel of claim 17, wherein the crosslinker comprises a diacrylate or dimethacrylate.

19. The hydrogel of claim 17, wherein the crosslinker comprises a polyalkylene oxide glycol diacrylate or a polyalkylene oxide glycol dimethacrylate.

20. The hydrogel of claim 17, wherein the crosslinker comprises N,N′-methylenebisacrylamide or N,N′-methylenebismethacrylamide.

21. The hydrogel of claim 2, wherein the (meth)acrylic monomer is acrylamide and the crosslinker is N,N′-methylenebisacrylamide.

22. The hydrogel of claim 2, wherein the radical initiator comprises an organic peroxide, an azo compound, or a persulfate.

23. The hydrogel of claim 2, wherein the multivalent cation is a divalent cation or a trivalent cation.

24. The hydrogel of claim 2, wherein the multivalent cation comprises Ca⁺², Mg⁺², Fe⁺², Fe⁺³, Zn⁺², Al⁺³, Cu⁺², Cu⁺³, a rare earth metal, or any combination thereof.

25. The hydrogel of claim 2, wherein the hydrogel comprises a multivalent cation and aminoglycoside antibiotic.

26. The hydrogel of claim 25, wherein the multivalent cation comprises Cu⁺² ions.

27. The hydrogel of claim 1, wherein the hydrogel further comprises a bioactive agent.

28. The hydrogel of claim 2, wherein the hydrogel further comprises a bioactive agent.

29. A microgel or nanogel comprising the hydrogel in any one of claims 1-28.

30. A pharmaceutical composition comprising the microgel or nanogel of claim 29 and a pharmaceutically-acceptable carrier.

31. A molded article comprising the hydrogel in any one of claims 1-28.

32. An adhesive hydrogel comprising (1) a layer comprising the hydrogel in any one of claims 1-27 having a first side and s second side, and (2) an adhesive layer adjacent to the first side of the hydrogel layer, wherein the adhesive comprises (a) a macromer comprising a plurality of phenolic groups covalently bonded to the macromer and (b) an enzyme for catalyzing covalent crosslinking between the phenolic groups in the macromer and phenolic groups present on a substrate.

33. The adhesive hydrogel of claim 32, wherein the phenolic groups have one hydroxyl group.

34. The adhesive hydrogel of claim 32, wherein the phenolic groups have two or more hydroxyl groups.

35. The adhesive hydrogel of claim 32, wherein the macromer is a peptide or protein.

36. The adhesive hydrogel of claim 32, wherein the macromer is a peptide or protein comprising one or more of tyrosine groups.

37. The adhesive hydrogel of claim 32, wherein the macromer is fulvic or humic acid.

38. The adhesive hydrogel of claim 32, wherein macromer is a synthetic polymer.

39. The adhesive hydrogel of claim 38, wherein the synthetic polymer comprises a polyacrylate comprising one or more pendant phenolic groups.

40. The adhesive hydrogel of claim 38, wherein the synthetic polymer comprises at least one fragment having the formula V 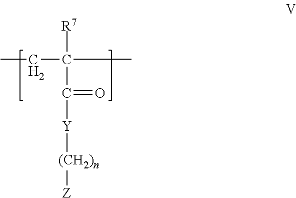
wherein R⁷ is hydrogen or an alkyl group;
n is from 1 to 10;
Y is oxygen, sulfur, or NR⁸, wherein R⁸ is hydrogen, an alkyl group, or an aryl group; and
Z is a phenolic group or a group comprising a phenolic group.

41. The adhesive hydrogel of claim 32, wherein the polymer comprises at least one dihydroxyl aromatic group capable of undergoing oxidation.

42. The adhesive hydrogel of claim 41, wherein the dihydroxyl aromatic group comprises a DOPA or a catechol moiety.

43. The adhesive hydrogel of claim 32, wherein the enzyme comprises a peroxidase or a catechol oxidase.

44. The adhesive hydrogel of claim 32, wherein the enzyme comprises horseradish peroxidase.

45. The adhesive hydrogel of claim 32, wherein the enzyme is physically entrapped within the macromer.

46. The adhesive hydrogel of claim 32, wherein the enzyme is covalently bonded to the macromer.

47. The adhesive hydrogel of claim 32, wherein the enzyme is physically entrapped within the macromer and covalently bonded to the macromer.

48. The adhesive hydrogel of claim 32, wherein the enzyme is covalently crosslinked to itself to form a self-crosslinked network.

49. The adhesive hydrogel of claim 32, wherein the enzyme is lyophilized prior to addition to the macromer.

50. The adhesive hydrogel of claim 32, wherein the adhesive layer further comprises a tackifier.

51. The adhesive hydrogel of claim 50, wherein the tackifier comprises an acrylic, a butyl rubber, ethylene-vinyl acetate, natural rubber, a nitrile, a silicone rubber, a styrene block copolymer, a vinyl ether, a glycosylated protein, a carbohydrate, or any combination thereof.

52. The adhesive hydrogel of claim 32, wherein the adhesive layer further comprises an enzyme stabilizer.

53. The adhesive hydrogel of claim 52, wherein the enzyme stabilizer is a sugar.

54. The adhesive hydrogel of claim 52, wherein the enzyme stabilizer is trehalose.

55. The adhesive hydrogel of claim 32, wherein the adhesive layer further comprises one or more bioactive agents.

56. The adhesive hydrogel of claim 32, wherein the adhesive layer further comprises silver ions entrapped within the adhesive layer and/or on the surface of the adhesive layer.

57. The adhesive hydrogel of claim 32, wherein the adhesive layer further comprises a peroxide source.

58. The adhesive hydrogel of claim 57, wherein the peroxide source comprises superoxide dismutase (SOD), glucose oxidase, or a combination thereof.

59. The adhesive hydrogel of claim 32, wherein a second adhesive layer is adhered to the second surface of the backing.

60. The adhesive hydrogel of claim 32, wherein a removable, protective layer is adjacent to the adhesive layer.

61. The adhesive hydrogel of claim 32, further comprising a backing on the second surface of the hydrogel layer.

62. The adhesive hydrogel of claim 61, wherein the backing comprises a water insoluble sheet or film, a woven fabric, a degradable film, a regenerated cellulose sheet, a decellularized tissue scaffold, a metal plate or a foil.

63. A method for adhering the adhesive hydrogel of claim 32 to a substrate having a first surface, the method comprising applying the adhesive hydrogel to the first surface of the substrate, wherein the adhesive layer on the adhesive hydrogel is in contact with the first surface of the substrate, and the first surface is wet with water.

64. The method of claim 63, wherein the substrate is bone, muscle, cartilage, ligaments, tendons, soft tissues, organs, or skin.

65. The method of claim 63, wherein the substrate is an implantable device.

66. The method of claim 63, wherein prior to applying the adhesive hydrogel to the first surface of the substrate, applying a macromer comprising a plurality of phenolic groups covalently bonded to the macromer to the first surface of the substrate.

67. The method of claim 63, wherein prior to applying the applying the adhesive hydrogel to the first surface of the substrate, applying a peroxide to the first surface of the substrate.

68. The method of claim 67, wherein the peroxide is hydrogen peroxide.

69. The method of claim 63, wherein prior to applying the adhesive hydrogel to the first surface of the substrate, (1) applying a macromer comprising a plurality of phenolic groups covalently bonded to the macromer to the first surface of the substrate followed by (2) applying a peroxide to the first surface of the substrate and macromer.

70. A kit comprising (1) the adhesive hydrogel of claim 32 and (2) a peroxide source.

71. An adhesive comprising (a) a macromer comprising a plurality of phenolic groups covalently bonded to the macromer and (b) an enzyme for catalyzing covalent crosslinking between the phenolic groups in the macromer and phenolic groups present on a substrate.
